(19) United States
(12) Patent Application Publication
(10) Pub. No.: US 2009/0069819 A1
(43) Pub. Date: Mar. 12, 2009

(54) COMPRESSIBLE DEPLOYMENT DEVICE

(76) Inventor: Aaron P. Barr, Fishers, IN (US)

Correspondence Address: RADER, FISHMAN & GRAUER PLLC, 39533 WOODWARD AVENUE, SUITE 140, BLOOMFIELD HILLS, MI, 48304-0610, US

(21) Appl. No.: 11/852,913

(22) Filed: Sep. 10, 2007

(51) Int. Cl. A61B 17/00

(52) U.S. Cl. 606/116

(57) ABSTRACT

A cannula for a marker deployment system is provided, including a tubular sidewall and at least one slit intersecting through the sidewall. The slit selectively permits at least a portion of the sidewall adjacent the slit to deform generally radially outward from an axis of the cannula.

### TECHNICAL FIELD

The present disclosure relates generally to site markers for breast biopsy procedures. More specifically, the present disclosure relates to a cannula for a marker deployment device including a tubular sidewall and at least one slit intersecting through the sidewall.

### BACKGROUND

In the diagnosis and treatment of breast cancer, it is often necessary to perform a biopsy to remove tissue samples from a suspicious mass. The suspicious mass is typically discovered during a preliminary examination involving visual examination, palpation, X-ray, magnetic resonance imaging (MRI), ultrasound imaging or other detection means.

When a suspicious mass is detected, a sample is taken by biopsy, and then tested to determine whether the mass is malignant or benign. This biopsy procedure can be performed by an open surgical technique, or through the use of a specialized biopsy instrument. To minimize surgical intrusion, a small specialized instrument such as a biopsy needle is inserted in the breast while the position of the needle is monitored using fluoroscopy, ultrasonic imaging, X-rays, MRI or other suitable imaging techniques.

Regardless of the method or instrument used to perform the biopsy, subsequent examination of the surgical site may be necessary, either in a follow up examination or for treatment of a cancerous lesion. Treatment often includes a mastectomy, lumpectomy, radiation therapy, or chemotherapy procedure that requires the surgeon or radiologist to direct surgical or radiation treatment to the precise location of the lesion. Because this treatment might extend over days or weeks after the biopsy procedure, and the original features of the tissue may have been removed or altered by the biopsy, it is desirable to insert a site marker into the surgical cavity to serve as a landmark for future identification of the location of the lesion.

However, some biopsy site markers may not be visible under all available modalities. When cancer is found at a biopsy site that has been previously marked with a site marker, the poor visibility of the biopsy site marker under ultrasound or other visualization modalities, may require that the patient undergo an additional procedure that places an additional device at the biopsy site to enable the surgeon to find the biopsy site in subsequent procedures. One known technique has been to place a breast lesion localization wire at the biopsy site. The localization wire is typically placed at the biopsy site via mammography and/or ultrasound.

Commonly assigned U.S. patent application Ser. No. 11/242,334 discloses a variety of markers. In some embodiments disclosed therein, expandable filament portions ‘hold’ a site marker in place within a biopsy cavity. That is, a site marker may include a bio-absorbable filament portion with a marker, where the marker is visible under multiple modalities and the filament portion will inhibit migration of the marker within the biopsy cavity. The filament portions of these structures typically define a site marker diameter that is greater than the outer diameter of the cannula. To insert a site marker within a biopsy site, the site marker is first compressed to a dimension that will permit the site marker to be interposed within the cannula. Next, the site marker is interposed within an opening of the cannula and the site marker and cannula are sterilized. The cannula is then inserted within the biopsy pathway such that the opening is positioned within the biopsy site, and the marker is deployed into the biopsy site. Once deployed, the site marker expands as the filament portions exit the cannula.

The site marker and delivery cannula must be sterile in order to be placed into a biopsy cavity. However, in some instances the filament portions, or other materials, plastically deform within the cannula due to the heat of sterilization, therefore the filament portions may not properly expand upon exiting the cannula. Lack of proper expansion may permit migration within the biopsy cavity.

Accordingly, there is a need for site markers and delivery systems that are compatible with sterilization and packaging techniques.

### BRIEF DESCRIPTION OF THE DRAWINGS

FIG. 1 is an exploded partially sectioned side view of a site marker deployment system.

FIG. 2 is a partial perspective view of a portion of the site marker deployment system of FIG. 1.

FIG. 3 is a partially sectioned side view of a portion of the site marker and the deployment device of FIG. 1.

FIG. 4 is a partially sectioned side view of the site marker and the deployment device of FIG. 1.

FIG. 5 is a partially sectioned side view of the site marker and the deployment device of FIG. 1.

FIG. 6 is a partially sectioned side view of the site marker and the deployment device of FIG. 1.

FIG. 7 is a partially sectioned side view of the site marker and the deployment device in packaging.

### DETAILED DESCRIPTION

Referring now to the discussion that follows and also to the drawings, illustrative approaches to the disclosed systems and methods are shown in detail. Although the drawings represent some possible approaches, the drawings are not necessarily to scale and certain features may be exaggerated, removed, or partially sectioned to better illustrate and explain the present invention. Further, the descriptions set forth herein are not intended to be exhaustive or otherwise limit or restrict the claims to the precise forms and configurations shown in the drawings and disclosed in the following detailed description.

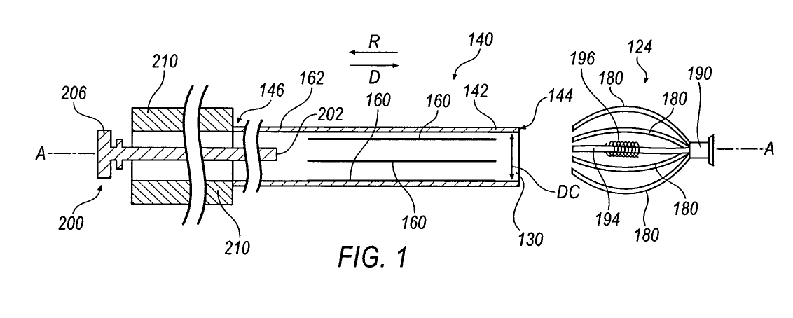

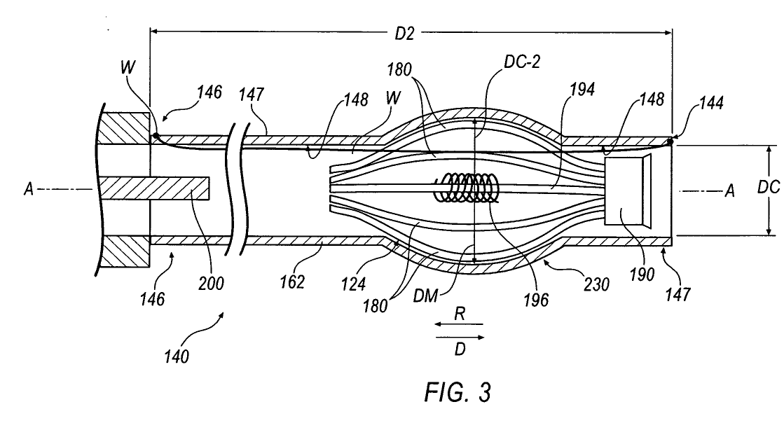

Turning now to the drawings and in particular to FIG. 1, an exemplar deployment device 140 is disclosed. In the illustration of FIG. 1, the deployment device 140 is used with a site marker 124. The deployment device 140 includes a cannula 142 that has an axis A-A and extends from a distal cannula end 144 to a proximal cannula end 146. At least a portion of the cannula 142 has a variable inner diameter that extends from a first inner diameter DC to a second inner dimension DC-2 (as shown in FIG. 3). The variable inner diameter will be explained in greater detail below. The distal cannula end 144 is generally defined by a distal end opening 130, having the first diameter DC. The deployment device 140 may also include a hub 210 that is selectively attached to the proximal cannula end 146. A push-rod 200 is at least partially disposed within the hub 210 and includes a push-rod distal end 202 and a push-rod proximal end 206.

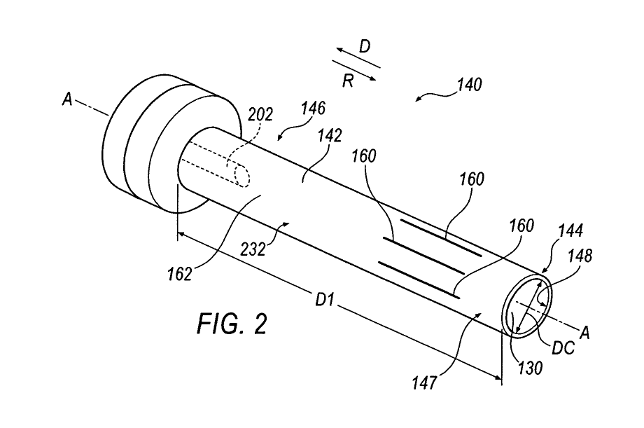

As best seen in FIGS. 1-2, the cannula 142 includes at least one slit 160 interposed between the distal cannula end 144 and the proximal cannula end 146 through the thickness of a tubular sidewall 162 of the cannula 142. The slits 160 are generally circumferentially spaced along the sidewall 162. In those embodiments that include more than one slit 160, at least a portion of one slit 160 is spaced in a circumferential direction from at least a portion of another slit 160 with respect to the axis A-A of the cannula 142. In the illustration as shown in FIGS. 1-2, the slits 160 are generally parallel to the axis A-A of the cannula 142 and equally spaced from one another. As illustrated, the slits 160 do not intersect the distal cannula end 144. It should be noted that while FIG. 1 illustrates two slits 160 through the sidewall 162, and FIG. 2 illustrates three slits 160 through the sidewall 162, any number of slits 160 may be included as well.

The deployment device 140 may be used with the site marker 124. The site marker 124 is elastically deformable such that the site marker 124 may be interposed through the distal opening 130 in a marker retracted configuration. As illustrated in FIG. 1, the site marker 124 includes a plurality generally elongated filament members 180 and at least one end connection 190. The filament members 180 are elastically deformable such that the site marker 124 can be compressed into the marker retracted configuration. The site marker 124 is elastically deformable to the marker retracted configuration such that the site marker 124 may be inserted into the cannula 142 prior to deployment.

In a representative embodiment as illustrated, the site marker 124 includes a filament member 194 having a marker element, or a permanent marker, 196 attached thereto. Alternatively the filament member could be a permanent element. It is understood that the filament member 194 and the permanent marker 196 may be omitted. It should also be noted that while FIG. 1 illustrates the site marker 124 including four filament members 180, any number of filament members 180 may be used as well.

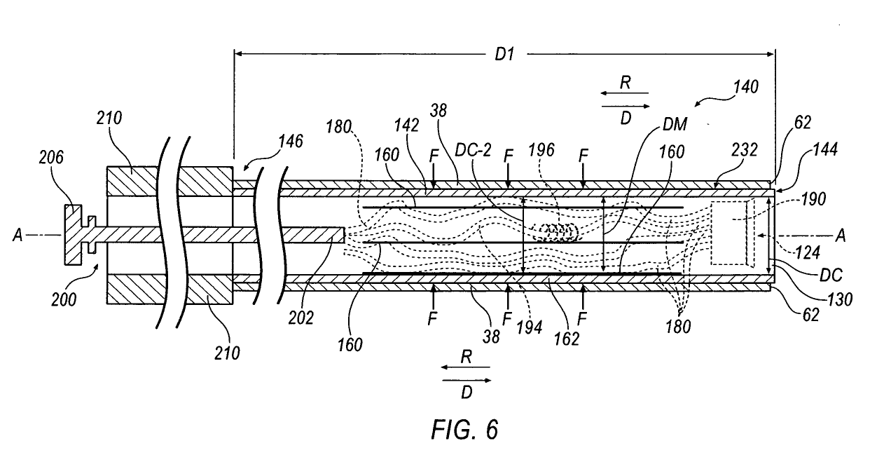

At least one of the filament members 180 serve to permit site marker 124 to be selectively configurable between a marker deployed configuration (FIG. 1) and the marker retracted configuration (FIG. 6). More specifically, as at least one of the filament members 180 is elastically deformed to configure the site marker 124 between the marker deployed configuration and the marker retracted configuration. In the marker deployed configuration, the site marker 124 can not be interposed through the cannula 142, by way of the distal opening 130.

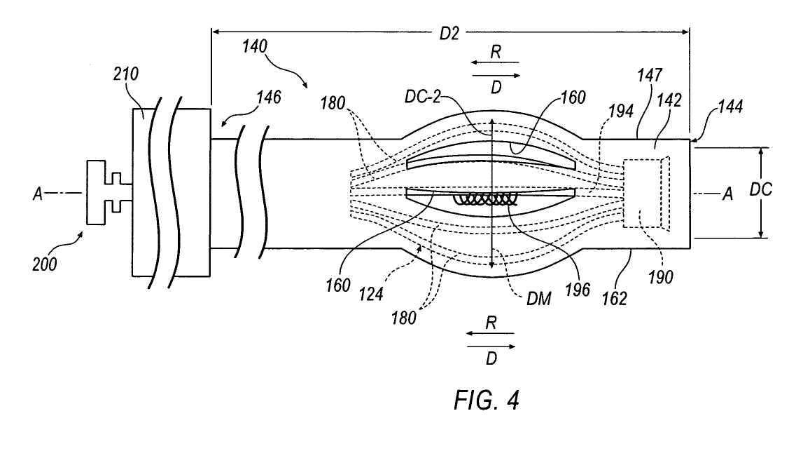

As best seen in FIGS. 3-4, the site marker 124 may also be configured into a marker retaining configuration that is defined by a first site marker dimension DM. The first site marker dimension I)M is measured generally normal to the axis A-A of the cannula 142. In one illustration, the first site marker dimension DM is about equal when the site marker 124 is in marker retaining configuration and the marker deployed configuration.

The deployment device 140 is selectively configurable between a deployed configuration and a protuberant configuration. FIGS. 1 and 2 illustrate the cannula 142 in the deployed configuration. In this configuration, the length of the cannula 142 is generally defined by a first distance D1 measured as the distance between the distal cannula end 144 and the proximal cannula end 146. When the cannula 142 is in the deployed configuration, the cannula 142 may be interposed within a lumen of an outer cannula, such as an outer cannula 38, as seen in FIG. 6. The deployment device also includes a sterilization configuration, which is discussed in greater detail below.

FIGS. 3 and 4 illustrate the cannula 142 in the protuberant configuration. The slits 160 intersect the sidewall 162 through a cannula inner surface 148 and a cannula outer surface 147 of the sidewall 162. The length of the cannula 142 is constructed from a flexible material such that the slits 160 permit cannula 142 to expand. When the cannula 142 is expanded, the second inner dimension DC-2 is about equal to the first site marker dimension IDM. The slits 160 selectively permit at least a portion of the sidewall 162 between the slits 160 to deform generally radially outward from the axis A-A of the cannula 142 to form a protuberance 230.

As best seen in FIG. 3, the site marker 124 may be at least partially interposed through the protuberance 230 interposed through the cannula 142 when the cannula 142 is in the protuberant configuration. The cannula 142 may be generally defined by a second distance, D2 which is measured as the distance between the distal cannula end 144 and the proximal cannula end 146. The second distance D2 may be less than the first inner distance D1. In one embodiment, the difference between the first inner distance D1 and the second distance D2 may range from about 0.25 cm to about 1.00 cm.

At least a portion of the sidewall 162 of the cannula 142 may define the protuberant configuration of FIGS. 3 and 4. When the cannula is in the protuberant configuration, the slits 160 extend generally radially outwardly away from the axis A-A. The cannula 142 is expanded to form the second inner dimension DC-2, and thereby allowing for the cannula 142 to encapsulate at least a portion of the site marker 124 when the site marker 124 is in the marker retaining or the marker retracted configuration. The protuberance 230 may be generally elliptical, and may be defined, at least in part, by the second inner dimension DC-2. The second inner dimension DC-2 may be defined by a diameter which is greater than the first inner diameter DC.

As seen in FIG. 3, the cannula 142 may be selectively deformable, and may also be retained in place by way of a retaining wire W. The retaining wire W held taught by being attached to both of the distal cannula end 144 and the proximal cannula end 146 of the cannula 142, thus retaining the cannula 142 in the protuberant configuration. The wire W exerts a compressive force against both of the distal cannula end 144 and the proximal cannula end 146, thereby retaining the cannula 142 in place in the protuberant configuration. Before the cannula 142 is selectively interposed within an outer cannula, such as the outer cannula 38, the wire W is detached from at least one of the distal cannula end 144 and the proximal cannula end 146. Although FIG. 3 illustrates the deployment device 140 with a wire W, it is understood that any retaining device, such as a plastic filament or a thread, may be used as well.

The wire W may be omitted, as illustrated in FIG. 4. Instead, the cannula 142 may be constructed from a material that allows for the cannula 142 to be selectively deformable. That is, the cannula 142 is selectively deformable by a selective inward radial force, being directed inwardly towards axis A-A of the cannula 142, thus urging at least a portion of the sidewall 162 into the deployment configuration. In this embodiment, the second distance D2 may be about equal to first inner distance, D1.

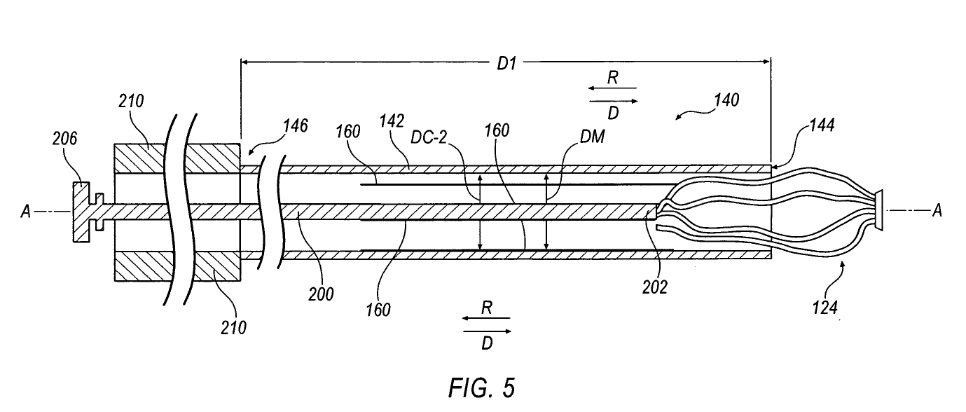

As best seen in FIG. 5-6 the push-rod 200 contacts a portion of the site marker 124 and is moved generally in the direction of the arrow D. The site marker 124 is urged out of the cannula 142 and is deployed into a desired location, such as a biopsy cavity, when the cannula 142 is in the deployment configuration.

FIG. 6 illustrates the cannula 142 interposed into an outer cannula, such as the outer cannula 38. At least a portion of the sidewall of the cannula 142, and more specifically the protuberance 230, is elastically deformed by a sidewall of the outer cannula 38. Thus, a selective inward radial force F deforms the protuberance 230 of the cannula 142, allowing for entry into the outer cannula 38.

During insertion of the cannula 142 into the outer cannula 38, the cannula 142 is urged into the deployment configuration. At least a portion of the sidewall 162 is directed radially inwardly towards the axis A-A of the cannula 142 when the sidewall 162 interferes with a distal end 62 of the outer cannula 38. More specifically, the filament members 180 of the site marker 124 are urged inwardly toward the axis A-A, and into the marker retracted position. When the deployment device 140 is in the sterilization configuration the site marker 124 can not be disengaged from the first inner diameter DC of the cannula 142.

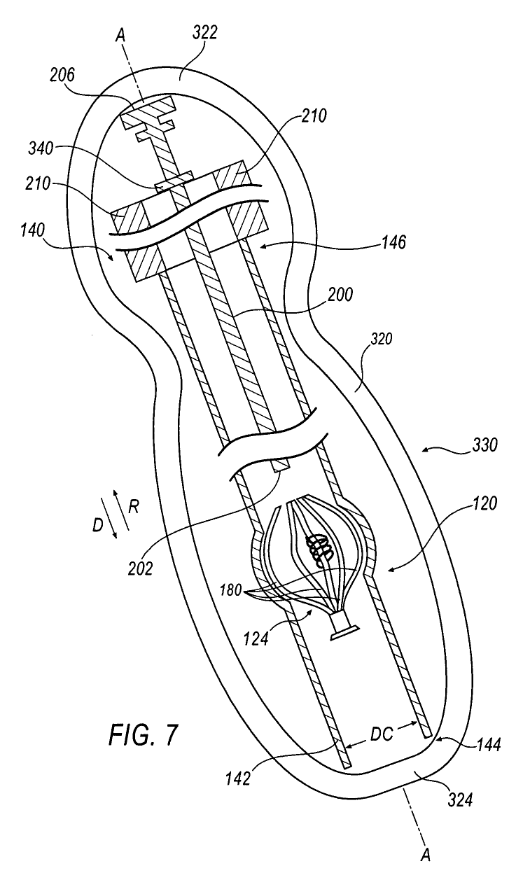

FIG. 7 illustrates the cannula 142 included as part of a system 120 that is supplied in a sterilization configuration. More specifically, in addition to the deployment configuration and the protuberant configuration, the deployment device 140 also includes the sterilization configuration. The deployment device 140 may be sterilized prior to deployment of the site marker 124. The push-rod 200 is selectively interposed within the cannula 142 just prior to deployment of the site marker 124, and the site marker 124 may be selectively axially moved relative to the cannula 142 by movement of the push-rod 200, and urged out of the distal end opening 130. The site marker 124 may be positioned between the distal cannula end 144 and the proximal cannula end 146. Thus positioned, the site marker 124 may be retained at least partially within the cannula 142.

Thus positioned, the site marker 124 may be sterilized while the filament members 180 are elastically deformed. Indeed, FIG. 7 illustrates the deployment device 140 positioned within a packaging 320. The push-rod 200 is held in place by a retaining ring 340. The retaining ring 340 maintains the push-rod in place such that the push-rod can not urge the site marker 124 out of the cannula 142 through the distal end 144, After the deployment device 140 is removed from the packaging 320 the retaining ring 340 may be detached from the push-rod 200.

In addition to the retaining ring 340, the packaging 320 includes a proximate retainer portion 322 and a distal retainer portion 324. In the embodiment illustrated, both of the proximate retainer portion 322 and the distal retainer portion 324 bindingly retain the cannula 142 and the push-rod 200 such that the push-rod 200 is prevented from moving in the direction D relative to the cannula 142. More importantly, the site marker 124 is retained in the sterilization configuration, until the deployment device 140 is removed from the packaging 320. Collectively, the site marker 124, the deployment device 140, and the packaging 320 comprise a system 330.

In general, the site markers described herein may be made from biocompatible materials such as, but not limited to, titanium, stainless steel, and platinum. These materials have appropriate densities for radiographic imaging, appropriate surface characteristics for ultrasonic imaging, and appropriate magnetic characteristics for magnetic resonance imaging. The site markers are preferably made from titanium; however, it is understood that any suitable biocompatible material may be used. Portions of the site markers may be made from bioabsorbable materials. Commonly owned U.S. patent application Ser. Nos. 11/242,334, 10/964,087 and 11/561,919 disclose a variety of site markers that may be used in conjunction with the deployment device 140.

Although the steps of the method of deploying the site markers described herein are listed in a preferred order, the steps may be performed in differing orders or combined such that one operation may perform multiple steps. Furthermore, a step or steps may be initiated before another step or steps are completed, or a step or steps may be initiated and completed after initiation and before completion of (during the performance of) other steps.

While the present disclosure has been particularly shown and described with reference to the foregoing preferred embodiments, it should be understood by those skilled in the art that various alternatives to the embodiments of the disclosure described herein may be employed in practicing the disclosure without departing from the spirit and scope of the disclosure as defined in the following claims. It is intended that the following claims define the scope of the disclosure embodiments within the scope of these claims and their equivalents be covered thereby. This description of the disclosure should be understood to include all novel and non-obvious combinations of elements described herein, and claims may be presented in this or a later application to any novel and non-obvious combination of these elements. The foregoing embodiment is illustrative, and no single feature or element is essential to all possible combinations that may be claimed in this or a later application.

What is claimed is:

1. A cannula for a marker deployment device, comprising:
a cannula having a tubular sidewall; and
at least one slit intersecting through the sidewall;
wherein the slit selectively permits at least a portion of the sidewall adjacent the slit to deform generally radially outward from an axis of the cannula.

2. The cannula as recited in claim 1, wherein the cannula includes a plurality of slits.

3. The cannula as recited in claim 2, wherein the slits are generally circumferentially spaced and at least a portion of one slit is spaced in a circumferential direction from at least a portion of another slit with respect to the axis of the cannula.

4. The cannula as recited in claim 1, wherein the slits are generally parallel to the axis of the cannula.

5. The cannula as recited in claim 1, wherein the cannula is selectively deformable between a deployment configuration and a protuberant configuration, the cannula being defined by a first distance between a proximal end and a distal end when in the deployment configuration and by a second distance between the proximal end and the distal end when in the protuberant configuration, the first distance being greater than the second distance.

6. The cannula as recited in claim 5, wherein the protuberant configuration is defined by at least a portion of the sidewall expanded radially outwardly to form a protuberance.

7. The cannula as recited in claim 5, wherein the cannula is selectively deformable by a selective inward radial force, the force being directed inwardly towards the axis of the cannula to urge at least a portion of the sidewall into the deployment configuration.

8. The cannula as recited in claim 5, wherein the protuberance is generally elliptical and is defined at least in part by a cannula inner dimension.

9. The cannula as recited in claim 8, wherein the cannula inner dimension is greater than a distal opening defined by a distal end inner diameter.

10. The cannula as recited in claim 5, wherein a site marker is selectively interposed through the cannula, and the site marker includes a first marker configuration and a second marker configuration, wherein at least a portion of the site marker is elastically deformed when the cannula is in the deployment configuration.

11. The cannula as recited in claim 10, wherein the site marker is able to be selectively interposed through the distal opening in the first marker configuration and the site marker is prevented from being interposed through a distal opening defined by a distal end inner diameter in the second marker configuration.

12. A site marker deployment system comprising:
a cannula including a sidewall, a distal end and a proximal end, and the distal end includes a distal opening generally defined by a distal end inner diameter;
at least two generally circumferentially spaced slits that intersecting the sidewall, wherein at least a portion of one slit is spaced in a circumferential direction from at least a portion of another slit with respect to an axis of the cannula; and
a site marker that is selectively interposed through the cannula, and the site marker includes a first marker configuration where the site marker is able to be selectively interposed through the distal end inner diameter, and a second marker configuration where the site marker is prevented from being interposed through the distal end inner diameter,
wherein the cannula is selectively deformable between a first distance between the proximal end and the distal end when in a deployed configuration and by a second distance between the proximal end and the distal end when in a protuberant configuration, the first distance being greater than the second distance;
wherein the protuberant configuration is defined by the slits selectively permitting at least a portion of the sidewall between the slits to deform generally radially outward from the axis of the cannula forming a protuberance of the sidewall between the ends;
wherein at least a portion of the site marker is elastically deformed when the cannula is in the deployed configuration.

13. The system as recited in claim 12, wherein the slits are generally parallel to the axis of the cannula.

14. The system as recited in claim 12, wherein the protuberance is generally elliptical and defined at least in part by a cannula inner dimension.

15. The system as recited in claim 14, wherein the cannula inner dimension is greater than the distal end inner diameter.

16. The system as recited in claim 12, wherein the cannula is selectively deformable by a selective inward radial force, the force being directed inwardly towards the axis of the cannula to urge at least a portion of the sidewall into the deployed configuration.

17. The system as recited in claim 12, wherein the site marker deployment system further comprises a push-rod for selectively axially moving the site marker relative to the cannula.

18. A method of deploying a site marker, comprising:
providing a site marker delivery system including a cannula and a site marker, the site marker delivery system including a proximal cannula end, a distal cannula end, a distal opening, a sidewall extending between the distal and proximal ends and at least one slit intersecting through the sidewall;
inserting the site marker though the distal opening of the cannula when the site marker is in a retracted configuration;
permitting at least a portion of the sidewall adjacent the slit to selectively deform generally radially outwardly from an axis of the cannula when the cannula is in a protuberant configuration; and
urging the site marker out of the cannula and into a biopsy cavity through the distal opening by a push-rod that is selectively interposed within the cannula;
wherein the site marker is selectively configurable between the retracted configuration where the site marker is able to be selectively interposed through the distal end opening, and a marker deployed configuration where the site marker is prevented from being interposed through the distal end inner diameter.

19. The method of claim 18, further comprising inserting the cannula into a separate outer cannula thereby deforming at least a portion of the side wall by a selective inward radial force, the force being directed generally radially inwardly towards the axis of the cannula.

20. The method of claim 19, wherein the cannula is in a deployed configuration when the cannula is interposed within the outer cannula.
